(19) United States
(12) Patent Application Publication
(10) Pub. No.: US 2019/0130358 A1
(43) Pub. Date: May 2, 2019

(54) SCREEN SHARING SYSTEM, METHOD, AND PROGRAM FOR REMOTE MEDICAL CARE

(71) Applicant: OPTIM CORPORATION, Shiga-shi, Shiga (JP)

(72) Inventor: Shunji SUGAYA, Tokyo (JP)

(73) Assignee: OPTiM Corporation, Saga-shi, Saga (JP)

(21) Appl. No.: 15/526,391

(22) PCT Filed: Jun. 30, 2016
(86) PCT No.: PCT/JP2016/069502

§ 371 Date: Jan. 4, 2018

(51) Int. Cl. G06Q 10/10; G16H 40/67

(52) U.S. Cl. CPC G06Q 10/105; G16H 40/67

(57) ABSTRACT

The invention is to appropriately change the doctor's hourly wage in the screen sharing system through which a doctor provides remote medical care to a patient by sharing a screen with the patient. The screen sharing system for remote medical care, includes a patient terminal 100 and a doctor terminal 200, the patient terminal 100 being connective with the doctor terminal 200 through a network, in which a doctor 600 provides remote medical care to a patient 500 while sharing the screen displaying the image of an affected area that is taken by the patient terminal 100 with the doctor terminal 200; a number-of-doctors detection module 321 that detects the number of doctors who can provide remote medical care; and a doctor's hourly-wage changing module 322 that changes the hourly wage of the doctor based on the detected number of the doctors.

### TECHNICAL FIELD

The present invention relates to a screen sharing system, a screen sharing method, and a screen sharing program for remote medical care to appropriately change the doctor's hourly wage in the screen sharing system through which a doctor provides remote medical care to a patient by sharing a screen with the patient.

### BACKGROUND ART

The home medical care and nursing system that reduces the load to which patients and caretakers who see a doctor regularly are subjected and processes medical care, data communication, billing, payment, etc., has been proposed (Patent Document 1).

### CITATION LIST

### Patent Literature

Patent Document 1: JP 2001-338068 A

### SUMMARY OF INVENTION

However, the system of Patent Document 1 is unsuitable to service to mediate between many patients and many doctors and offer necessary remote medical care and health consultations to the patients any time regardless of their addresses because the system is required to be introduced into specific medical institutions.

To render such a service to mediate between many patients and many doctors and offer necessary remote medical care and health consultations to the patients any time, the system needs to always secure the doctors who can provide medical care. To accomplish this, appropriate payment to the doctors has to be determined. However, in the existing system, the method for determining payment to the doctors is not disclosed.

In view of the above-mentioned problems, an objective of the present invention is to provide a screen sharing system, a screen sharing method, and a screen sharing program for remote medical care to appropriately change the doctor's hourly wage in the screen sharing system through which a doctor provides remote medical care to a patient by sharing a screen with the patient.

The first aspect of the present invention provides a screen sharing system for remote medical care that changes a doctor's hourly wage based on the number of doctors who are connective to the system.

According to the first aspect of the present invention, a screen sharing system for remote medical care changes a doctor's hourly wage based on the number of doctors who are connective to the system.

The first aspect of the present invention falls into the category of a screen sharing system for remote medical care, but the categories of a screen sharing method and a screen sharing program for remote medical care have similar functions and effects.

The second aspect of the present invention provides the screen sharing system according to the first aspect of the present invention that changes a doctor's hourly wage based on a time period.

According to the second aspect of the present invention, the screen sharing system according to the first aspect of the present invention changes a doctor's hourly wage based on a time period.

The third aspect of the present invention provides the screen sharing system according to the first or the second aspect of the present invention that changes a doctor's hourly wage based on the number of patients who are connecting to the system.

According to the third aspect of the present invention, the screen sharing system according to the first or the second aspect of the present invention changes a doctor's hourly wage based on the number of patients who are connecting to the system.

The fourth aspect of the present invention provides the screen sharing system according to any one of the first to the third aspects of the present invention that displays the doctor's hourly wage on a doctor terminal.

According to the fourth aspect of the present invention, the screen sharing system according to any one of the first to the third aspects of the present invention displays the doctor's hourly wage on a doctor terminal.

The fifth aspect of the present invention provides the screen sharing system according to any one of the first to the fourth aspects of the present invention that displays the number of doctors who are connective to the system on a doctor terminal.

According to the fifth aspect of the present invention, the screen sharing system according to any one of the first to the fourth aspects of the present invention displays the number of doctors who are connective to the system on a doctor terminal.

The sixth aspect of the present invention provides a screen sharing method for remote medical care, including a step of changing a doctor's hourly wage based on the number of doctors who are connective to the system.

The seventh aspect of the present invention provides a computer program product for use in a screen sharing system for remote medical care, including a non-transitory computer usable medium having a set of instructions physically embodied therein, the set of instructions including computer readable program code, which when executed by the system causes the information processing unit to change a doctor's hourly wage based on the number of doctors who are connective to the system.

The eighth aspect of the present invention provides a screen sharing system for remote medical care, including:

a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area;

a number-of-doctors detection unit that detects the number of doctors who can provide remote medical care based on the number of doctor terminals that are connective to the system; and

a doctor's hourly-wage changing unit that changes the hourly wage of the doctor based on the detected number of the doctors.

According to the eighth aspect of the present invention, a screen sharing system for remote medical care, includes:

a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area;

a number-of-doctors detection unit that detects the number of doctors who can provide remote medical care based on the number of doctor terminals that are connective to the system; and

a doctor's hourly-wage changing unit that changes the hourly wage of the doctor based on the detected number of the doctors.

The eighth aspect of the present invention falls into the category of a screen sharing system for remote medical care, but the categories of a screen sharing method and a screen sharing program for remote medical care have similar functions and effects.

The ninth aspect of the present invention provides the screen sharing system according to the eighth aspect of the present invention, in which the doctor's hourly-wage changing unit changes a doctor's hourly wage based on a time period.

According to the ninth aspect of the present invention, the screen sharing system according to the eighth aspect of the present invention, in which the doctor's hourly-wage changing unit changes a doctor's hourly wage based on a time period.

The tenth aspect of the present invention provides the screen sharing system according to the eighth or the ninth aspect of the present invention, further including a number-of-patients counting unit that counts the number of patients who are connecting to the system, in which the doctor's hourly-wage changing unit changes the hourly wage of the doctor based on the number of the patients.

According to the tenth aspect of the present invention, the screen sharing system according to the eighth or the ninth aspect of the present invention further includes a number-of-patients counting unit that counts the number of patients who are connecting to the system, in which the doctor's hourly-wage changing unit changes the hourly wage of the doctor based on the number of the patients.

The eleventh aspect of the present invention provides the screen sharing system according to any one of the eighth to the tenth aspects of the present invention, further including a doctor's hourly-wage display unit that displays the doctor's hourly wage on the doctor terminal.

According to the eleventh aspect of the present invention, the screen sharing system according to any one of the eighth to the tenth aspects of the present invention further includes a doctor's hourly-wage display unit that displays the doctor's hourly wage on the doctor terminal.

The twelfth aspect of the present invention provides the screen sharing system according to any one of the eighth to the eleventh aspects of the present invention, further including a number-of-doctors display unit that displays the number of the doctors on the doctor terminal.

According to the twelfth aspect of the present invention, the screen sharing system according to any one of the eighth to the eleventh aspects of the present invention further includes a number-of-doctors display unit that displays the number of the doctors on the doctor terminal.

The thirteenth aspect of the present invention provides a screen sharing method for remote medical care, executed by a screen sharing system for remote medical that includes a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area; including the steps of:

detecting the number of doctors who can provide remote medical care based on the number of doctor terminals that are connective to the system; and

changing the hourly wage of the doctor based on the detected number of the doctors.

The fourteenth aspect of the present invention provides a computer program product for use in a screen sharing system for remote medical care that includes a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area, including a non-transitory computer usable medium having a set of instructions physically embodied therein, the set of instructions including computer readable program code, which when executed by the system causes the information processing unit to:

detect the number of doctors who can provide remote medical care based on the number of doctor terminals that are connective to the system; and

change the hourly wage of the doctor based on the detected number of the doctors.

The present invention can provide a screen sharing system, a screen sharing method, and a screen sharing program for remote medical care to appropriately change the doctor's hourly wage in the screen sharing system through which a doctor provides remote medical care to a patient by sharing a screen with the patient.

### BRIEF DESCRIPTION OF DRAWINGS

FIG. 1 shows a schematic diagram of a preferable embodiment of the present invention.

FIG. 2 shows a functional block diagram of the patient terminal 100, the doctor terminal 200, and the server 300 to illustrate the relationship among the functions.

FIG. 3 shows a flow chart of the doctor's hourly-wage changing process based on the detection of the number of doctors that is performed by the doctor terminal 200 and the server 300.

FIG. 4 shows a flow chart of the doctor's hourly-wage changing process based on the detection of the number of doctors and a time period that is performed by the doctor terminal 200 and the server 300.

FIG. 5 shows a flow chart of the screen sharing process for remote medical care performed by the patient terminal 100, the doctor terminal 200, and the server 300.

FIG. 6 shows a functional block diagram of the patient terminal 100, the doctor terminal 200, and the server 300 to illustrate the relationship among the functions if the system is provided with a number-of-patients counting function.

FIG. 7 shows a flow chart of the doctor's hourly-wage changing process based on the detection of the number of doctors and the count of the number of the patients that is performed by the doctor terminal 200 and the server 300.

FIG. 8 shows a functional block diagram of the patient terminal 100, the doctor terminal 200, and the server 300 to illustrate the relationship among the functions if the system has a doctor's hourly-wage display function and a number-of-doctors display function.

FIG. 9 shows a flow chart of the doctor's hourly-wage changing process based on the detection of the number of doctors that is performed by the doctor terminal 200 and the server 300 if a doctor's hourly wage display process and a number-of-doctors display process are performed.

FIG. 10 shows an output example of the patient terminal 100 and the doctor terminal 200 to provide medical care through the screen sharing system for remote medical care.

FIG. 11 shows an example illustrating the correspondence of the number of doctors to an hourly wage.

FIG. 12 shows an example illustrating the correspondence of the number of doctors to a time period.

FIG. 13 shows an example illustrating the correspondence of the number of doctors and the number of patients to an hourly wage.

FIG. 14 shows an output example to display the number of doctors and the hourly wage on the doctor terminal 200 during medical care.

FIG. 15 shows an output example to have a doctor 600 to log in the system after the doctor 600 confirms the number of doctors and the hourly wage that are being displayed on the doctor terminal 200.

### DESCRIPTION OF EMBODIMENTS

Embodiments of the present invention will be described below with reference to the attached drawings. However, these are illustrative only, and the technological scope of the present invention is not limited thereto.

### Overview of Screen Sharing System for Remote Medical Care

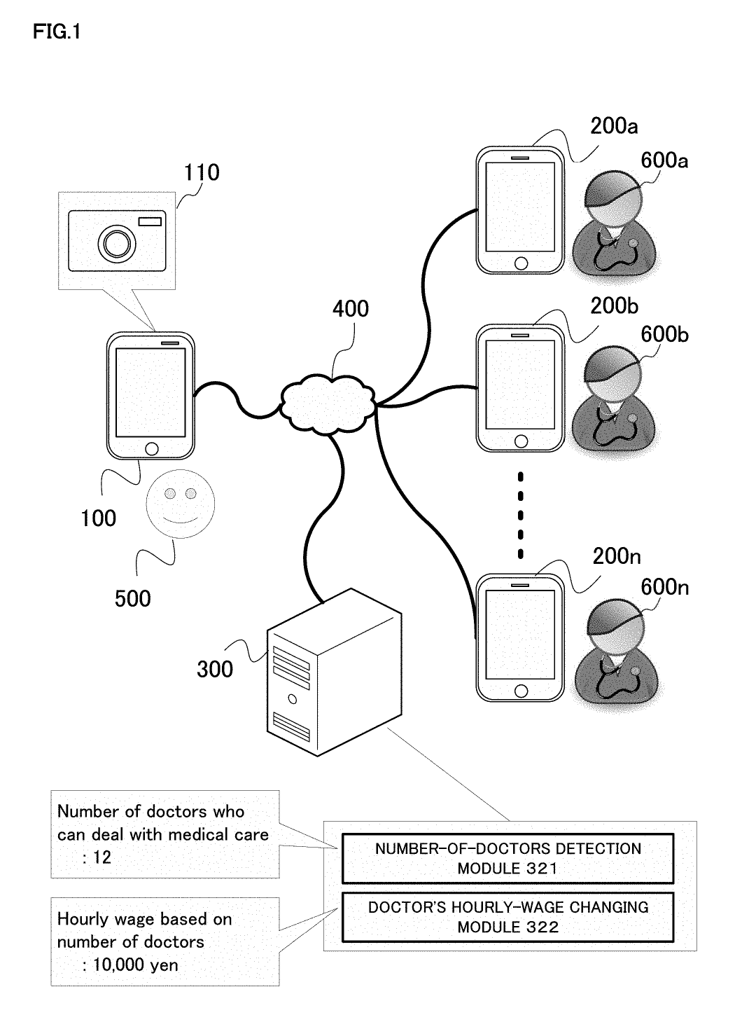

FIG. 1 shows a schematic diagram of a preferable embodiment of the present invention. The overview of the present invention will be described below with reference to FIG. 1.

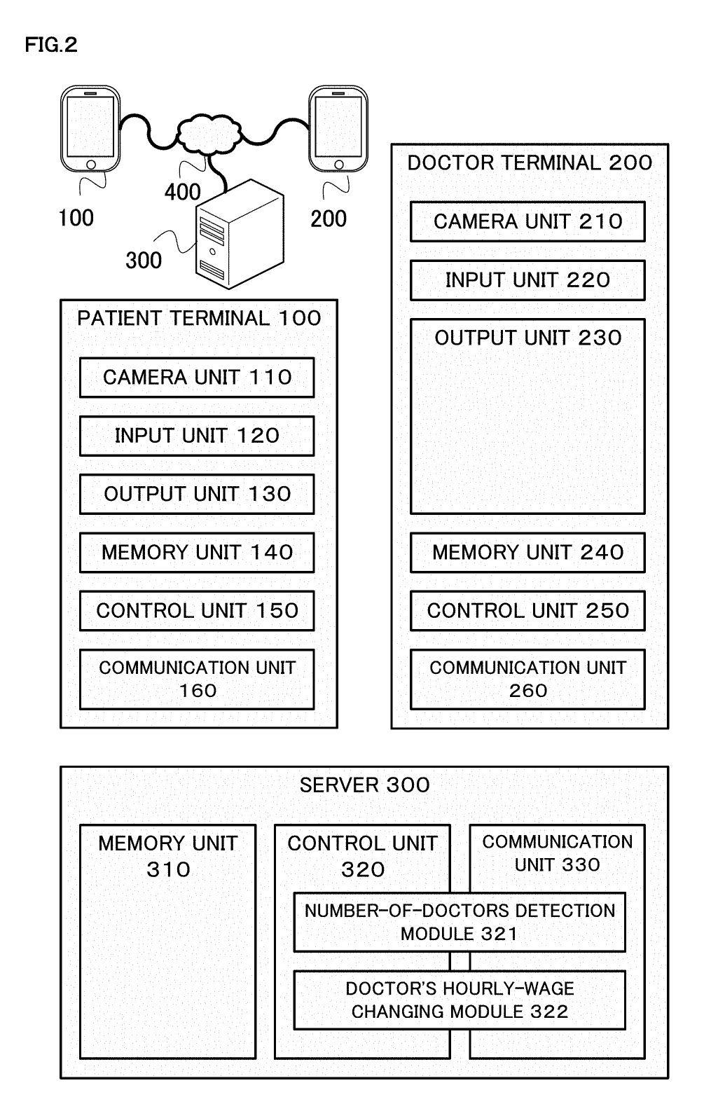

The patient terminal 100, the doctor terminals 200a to 200n, and the server 300 can be connective among these through a communication network 400. A patient 500 is assumed to have the patient terminal 100. Doctors 600a to 600n are assumed to have the doctor terminals 200a to 200n, respectively. The patient terminal 100 includes a camera unit 110, an input unit 120, an output unit 130, a memory unit 140, a control unit 150, and a communication unit 160 as shown in FIG. 2. The doctor terminals 200a to 200n each include a camera unit 210, an input unit 220, an output unit 230, a memory unit 240, a control unit 250, and a communication unit 260 as shown in the doctor terminal 200 of FIG. 2. The server 300 includes a memory unit 310, a control unit 320, and a communication unit 330 as shown in FIG. 2. The control unit 320 achieves a number-of-doctors detection module 321 and a doctor's hourly-wage changing module 322 in cooperation with the communication unit 330. The communication network 400 may be a public line network such as the Internet or a private line network.

The patient terminal 100 and the doctor terminals 200a to 200n may each be a general information terminal that is capable to share their screen and execute various applications. These terminals are each an information device or an electrical appliance with the functions to be described later. For example, the terminals may each be a general information appliance such as a mobile phone, a smart phone, a tablet PC, a notebook, or a wearable device, a complex printer, a television, and a network device such as a router or a gateway. The terminals may also each be a white good such as a refrigerator or a washing machine. The terminals may also each be an electrical appliance such as a telephone, a netbook terminal, a slate terminal, an electronic book terminal, an electronic dictionary terminal, a portable music player, and a portable content player and recorder. The smart phones shown as the patient terminal 100 and the doctor terminals 200a to 200n in the attached drawings are just one example. FIG. 1 shows only one patient terminal 100 and one patient 500. However, the screen sharing system for remote medical care has no problem even if connecting with a plurality of patients 500a to 500m and a plurality of patient terminals 100a to 100m at the same time.

In the screen sharing system for remote medical care shown in FIG. 1, a selected doctor 600 provides remote medical care to a patient 500 while sharing the screen of the patient terminal 100 that displays the image of the affected area that is taken by the camera 110 of the patient terminal 100 with the doctor terminal 200 of the doctor 600.

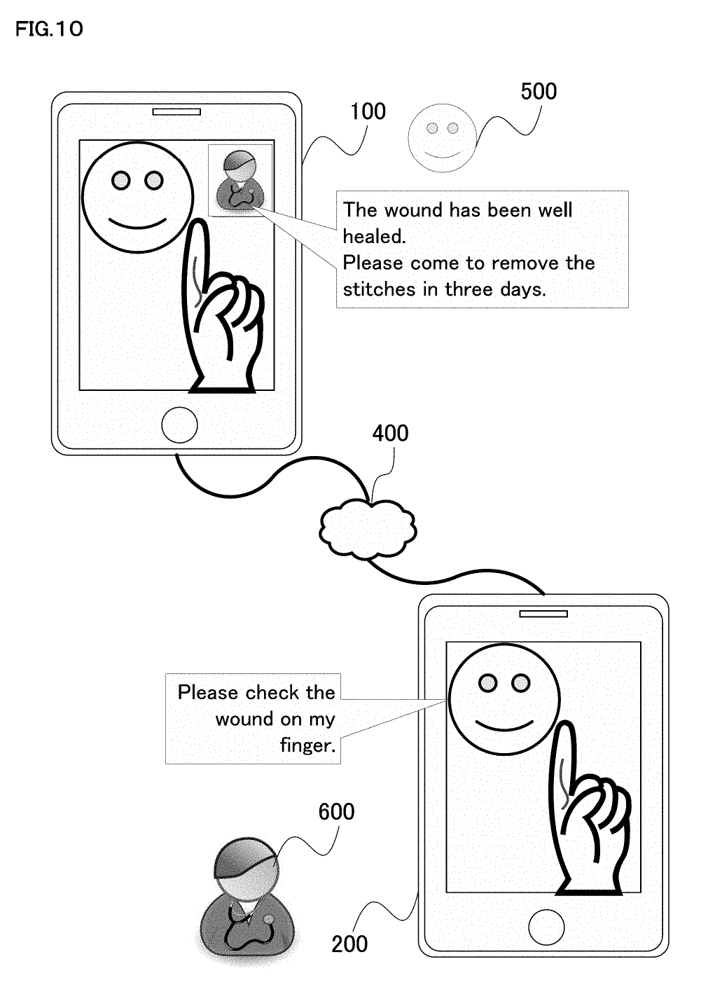

FIG. 10 shows an output example of the patient terminal 100 and the doctor terminal 200 if medical care is provided from the screen sharing system for remote medical care. A patient 500 takes an image of the affected area with the camera unit 110 of the patient terminal 100 and requests medical care from a doctor 600. The doctor 600 checks the affected area of the patient 500 with the doctor terminal 200, requests the patient 500 to take an image of the affected area from another angle, and instructs the patient 500 to come to the hospital as needed. The image of the doctor 600 taken by the camera unit 210 of the doctor terminal 200 may be displayed on the output unit 130 of the patient terminal 100. Sharing the image of a doctor 600 with a patient 500 can provide a sense of ease with the patient 500 more than providing medical care only by voice. As not shown in FIG. 10, an image taken by the doctor terminal 200 may be displayed on the camera unit 210 for doctor's 600 confirmation.

When a doctor 600 logs in the screen sharing system for remote medical care, the number-of-doctors detection module 321 of the server 300 detects the connected doctor terminal 200 and updates the number of doctors being connected. Likewise, when a doctor 600 logs out of the screen sharing system for remote medical care, the number-of-doctors detection module 321 detects the unconnected doctor terminal 200 and updates the number of doctors being connected. In consideration of the case where the connection with a doctor terminal 200 is unexpectedly lost, it is preferable that the number-of-doctors detection module 321 check whether or not the communication with the doctor terminals 200 that should be connected are alive at regular intervals and update the number of doctors being connected.

The doctor's hourly-wage changing module 322 of the server 300 changes the doctor's hourly wage based on the number of doctors whom the number-of-doctors detection module 321 has detected.

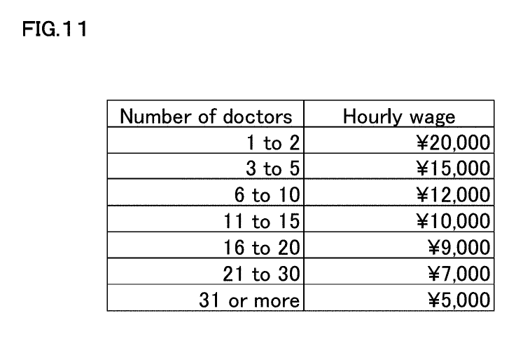

FIG. 11 shows an example illustrating the correspondence of the number of doctors to an hourly wage. The server 300 is assumed to store data that show the correspondence of the number of doctors to an hourly wage in the memory unit 310. If the number of doctors who are being connected and able to provide medical care is provided, the doctor's hourly wage can be calculated by looking up the table shown in FIG. 11. If the number-of-doctors detection module 321 detects twelve doctors who can provide medical care, the doctor's hourly-wage changing module 322 determines the doctor's hourly wage as 10,000 yen depending on the number of doctors: 12. Since the table shown in FIG. 11 is just an example, the correspondence of the number of doctors to an hourly wage that are applied to the system may be changed depending on the number of all the doctors registered in the screen sharing system for remote medical care. The doctor's hourly-wage changing module 322 may automatically create the correspondence table of the number of doctors to an hourly wage based on the budget allocated to the doctors' wages. Furthermore, the doctor's hourly-wage changing module 322 may mechanically learn the settings of the correspondence of the number of doctors to an hourly wage when the rate of doctors who are working on the system is high, when a patient is more likely to receive medical care immediately, and when the doctor's wage decreases, and may calculate based on the result.

As described above, the present invention relates to a screen sharing system, a screen sharing method, and a screen sharing program for remote medical care to appropriately change the doctor's hourly wage depending on the number of doctors who are communicative to the screen sharing system through which a doctor provides remote medical care to a patient by sharing a screen with the patient.

### Functions

FIG. 2 shows a functional block diagram of the patient terminal 100, the doctor terminal 200, and the server 300 to illustrate the relationship among the functions. The patient terminal 100, the doctor terminal 200, and the server 300 can be connective among these through a communication network 400. The patient terminal 100 includes a camera unit 110, an input unit 120, an output unit 130, a memory unit 140, a control unit 150, and a communication unit 160. The doctor terminal 200 includes a camera unit 210, an input unit 220, an output unit 230, a memory unit 240, a control unit 250, and a communication unit 260. The server 300 includes a memory unit 310, a control unit 320, and a communication unit 330. The control unit 320 achieves a number-of-doctors detection module 321 and a doctor's hourly-wage changing module 322 in cooperation with the communication unit 330. The communication network 400 may be a public line network such as the Internet or a private line network.

The patient terminal 100 and the doctor terminal 200 may each be a general information terminal that is capable to share their screen and execute various applications. These terminals are each an information device or an electrical appliance with the functions to be described later. For example, the patient terminal 100 and the doctor terminal 200 may each be a general information appliance such as a mobile phone, a smart phone, a tablet PC, a notebook, or a wearable device, a complex printer, a television, and a network device such as a router or a gateway. These terminals may also each be a white good such as a refrigerator or a washing machine. These terminals may also each be an electrical appliance such as a telephone, a netbook terminal, a slate terminal, an electronic book terminal, an electronic dictionary terminal, a portable music player, and a portable content player and recorder. The smart phones shown as the patient terminal 100 and the doctor terminal 200 in the attached drawings are just one example. FIG. 2 shows only one patient terminal 100 and one doctor terminal 200. However, the screen sharing system for remote medical care has no problem even if connecting with a plurality of doctors terminal 200a to 200m and a plurality of patient terminals 100a to 100m at the same time.

The patient terminal 100 includes a camera in the camera unit 110. The patient terminal 100 takes and converts an image into digital data and stores the digital data in the memory unit 140. The image may be a still image or a moving image. If the image is a moving image, the control unit 150 can capture a part of the moving image to store in the memory unit 140 as a still image. The obtained taken image is an accurate image with information as much as needed for medical care provided from the doctor terminal 200. The pixel count and the image quality can be specified.

The input unit 120 has functions necessary for imaging instruction and connection to the screen sharing system for remote medical care. The input unit 110 may include a liquid crystal display to achieve a touch panel function, a key board, a mouse, a pen tablet, and a hardware button on the device, and a microphone to perform voice recognition. The features of the present invention are not limited in particular by an input method.

The output unit 130 has functions necessary to display the taken images and share a screen with the doctor terminal 200. The output unit 130 may take forms such as a liquid crystal display, a PC display, and a projector to display images and output voices. The features of the present invention are not limited in particular by an output method.

The memory unit 140 includes a data storage unit such as a hard disk or a semiconductor memory to store moving and still images that have been taken, information on the settings when images are taken, data necessary for a patient 500 to use the screen sharing system for remote medical care, and the like.

The control unit 150 includes a central processing unit (hereinafter referred to as “CPU”), a random access memory (hereinafter referred to as “RAM”), and a read only memory (hereinafter referred to as “ROM”).

The communication unit 160 includes a device that is communicative to other devices, such as a Wireless Fidelity (Wi-Fi®) enabled device complying with, for example, IEEE 802.11, or a wireless device complying with the IMT-2000 standard such as the third and the fourth generation mobile communication systems. The communication unit may include a wired device for LAN connection.

The doctor terminal 200 preferably includes a camera in the camera unit 210. The doctor terminal 200 takes and converts an image into digital data and stores the digital data in the memory unit 240. The image may be a still image or a moving image. If the image is a moving image, the control unit 250 can capture a part of the moving image to store in the memory unit 240 as a still image. The obtained taken image is an accurate image with information as much as needed for a patient to display the image on the patient terminal 100. The pixel count and the image quality can be specified.

The input unit 220 has functions necessary for imaging instruction and connection to the screen sharing system for remote medical care. The input unit 110 may include a liquid crystal display to achieve a touch panel function, a key board, a mouse, a pen tablet, and a hardware button on the device, and a microphone to perform voice recognition. The features of the present invention are not limited in particular by an input method.

The output unit 230 has functions necessary to display the obtained taken images and share a screen with the patient terminal 100. The output unit 230 may take forms such as a liquid crystal display, a PC display, and a projector to display images and output voices. The features of the present invention are not limited in particular by an output method.

The memory unit 240 includes a data storage unit such as a hard disk or a semiconductor memory to store moving and still images that have been taken, information on the settings when images are taken, data necessary for a doctor 600 to use the screen sharing system for remote medical care, and the like.

The control unit 250 includes a CPU, a RAM, and a ROM.

The communication unit 260 includes a device that is communicative to other devices, such as a Wi-Fi® enabled device complying with, for example, IEEE 802.11, or a wireless device complying with the IMT-2000 standard such as the third and the fourth generation mobile communication systems. The communication unit may include a wired device for LAN connection.

The server 300 may be a general server provided with the functions to be described later.

The server 300 also includes a storage unit as the memory unit 310 such as a hard disk or a semiconductor memory to store data. The memory unit 310 stores information on a patient terminal 100, a patient 500, and a doctor terminal 200, and a doctor 600, data on the correspondence of number of doctors to a hourly wage, and the like.

The server 300 includes a control unit 320 provided with a CPU, a RAM, a ROM, etc.

The communication unit 330 is communicative to other devices through a wired or unwired network. The communication unit 330 includes a Wi-Fi® enabled device complying with, for example, IEEE 802.11, or a wireless device complying with the IMT-2000 standard such as the third and the fourth generation mobile communication systems. The communication unit may include a wired device for LAN connection.

In the server 300, the control unit 320 reads a predetermined program to achieve a number-of-doctors detection module 321 and a doctor's hourly-wage changing module 322 in cooperation with the communication unit 330.

### Doctor's Hourly-Wage Changing Process Based on Detection of Number of Doctors

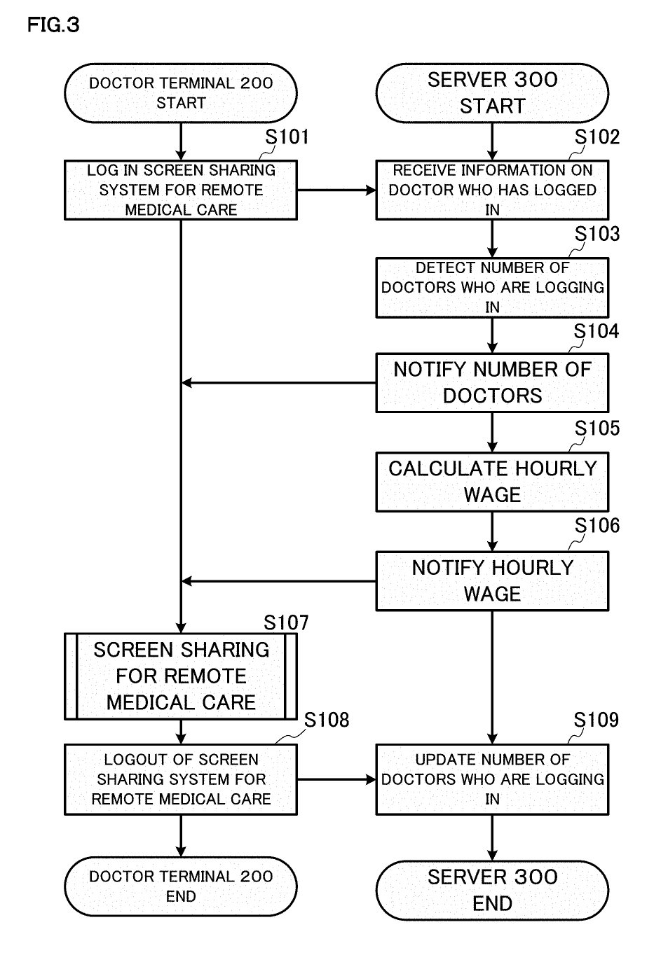

FIG. 3 shows a flow chart of the doctor's hourly-wage changing process based on the detection of the number of doctors that is performed by the doctor terminal 200 and the server 300. The tasks executed by the modules of each of the above-mentioned units will be explained below together with this process. The patient terminal 100, the doctor terminal 200, and the server 300 can be connective among these through a communication network 400. The communication network 400 may be a public line network such as the Internet or a private line network.

First, the doctor terminal 200 transmits doctor information necessary for login to the server 300 through the communication unit 260 and then logs in the screen sharing system for remote medical care (Step S101). The doctor information herein consists of account information including a name, a nickname, and a doctor ID, authentication information including a password, a passphrase, an IC card, a smart card, a fingerprint, and an iris, and the like. The flow chart shown in FIG. 3 illustrates that the login is succeeded for shorthand. However, if the login fails, the login process may suggest to the doctor to try the login again. If the login fails a certain number of times, the login process may freeze the account and then ends.

Then, the server 300 receives doctor information on the doctor who has newly logged in through the communication unit 330 (Step S102). The server 300 receives not only doctor information but also information necessary to check the connection with the doctor terminal 200 in the following process from the doctor terminal 200.

Then, the number-of-doctors detection module 321 detects the number of doctors who are logged in the screen sharing system for remote medical care (Step S103). At this point, the number-of-doctors detection module 321 detects a doctor terminal 200 that has been newly connected and performs a process to update the number of doctors being connected. At this time, in consideration of the case where the connection with a logged in doctor terminal 200x is unexpectedly lost, the number-of-doctors detection module 321 may check whether or not the communication with the doctor terminals that should be connected are alive at regular intervals and may detect the number of doctors being connected.

Then, the number-of-doctors detection module 321 notifies the number of doctors to the doctor terminal 200 (Step S104). The notified number of doctors may be used when the number of doctors being connected is confirmed on the doctor terminal 200. The number-of-doctors detection module 321 may notify the number of doctors to the doctor terminals 200a to 200n other than the doctor terminal 200 that are logged in the screen sharing system for remote medical care, at the same time. FIG. 3 shows a flowchart illustrating that the number-of-doctors detection module 321 of the server 300 voluntarily notifies the number of doctors. However, the number-of-doctors detection module 321 may notify the number of doctors in response to a query about the number of doctors from the doctor terminal 200.

The doctor's hourly-wage changing module 322 calculates the doctor's hourly wage based on the number of doctors who are logged in the screen sharing system for remote medical care that is detected by the number-of-doctors detection module 321 (Step S105).

FIG. 11 shows an example illustrating the correspondence of the number of doctors to an hourly wage. The server 300 is assumed to store data that show the correspondence of the number of doctors to an hourly wage in the memory unit 310. If the number of doctors who are being connected and able to provide medical care is provided, the doctor's hourly wage can be calculated by looking up the table shown in FIG. 11. If the number-of-doctors detection module 321 detects four doctors who can provide medical care, the doctor's hourly-wage changing module 322 determines the doctor's hourly wage as 15,000 yen depending on the number of doctors: 4. Since the table shown in FIG. 11 is just an example, the correspondence of the number of doctors to an hourly wage applied to the system may be changed depending on the number of all the doctors registered in the screen sharing system for remote medical care. The doctor's hourly-wage changing module 322 may automatically create the correspondence table of the number of doctors to an hourly wage based on the budget allocated to the doctors' wages. Furthermore, the doctor's hourly-wage changing module 322 may mechanically learn the settings of the correspondence of the number of doctors to an hourly wage when the rate of doctors who are working on the system is high, when a patient is more likely to receive medical care immediately, and when the doctor's wage decreases, and may calculate based on the result.

Then, the doctor's hourly-wage changing module 322 notifies the hourly wage to the doctor terminal 200 (Step S106). The notified hourly wage may be used when the hourly wage is confirmed on the doctor terminal 200. The doctor's hourly-wage changing module 322 may notify the hourly wage to the doctor terminals 200a to 200n other than the doctor terminal 200 that are logged in the screen sharing system for remote medical care, at the same time. FIG. 3 shows a flowchart illustrating that the doctor's hourly-wage changing module 322 of the server 300 voluntarily notifies the hourly wage. However, the doctor's hourly-wage changing module 322 may notify the hourly wage in response to a query about the hourly wage from the doctor terminal 200. If the hourly wage changes every a few minutes, the calculation of the wage of the doctor 600 is complicated. Therefore, the update timing of the hourly wage to the doctor terminal 200 may be set in the system at every preferable period, for example, 10, 30, or 60 minutes

Then, the doctor terminal 200 performs the screen sharing process for remote medical care with the patient terminal 100 (Step S107). The screen sharing process for remote medical care will be described later.

After the screen sharing process for remote medical care ends, the doctor terminal 200 transmits instruction to log out of the screen sharing system for remote medical care to the server 300 through the communication unit 260 (Step S108).

Finally, the server 300 receives information on the doctor who has logged out through the communication unit 330. Then, the number-of-doctors detection module 321 updates the number of doctors who are logged in the screen sharing system for remote medical care (Step S109). At this point, the number-of-doctors detection module 321 performs a process to decrease the number of doctors being connected by the number of doctor terminals 200 that has been disconnected by logout. At this time, in consideration of the case where the connection with a logged in doctor terminal 200x is unexpectedly lost, the number-of-doctors detection module 321 may check whether or not the communication with the doctor terminals that should be connected are alive at regular intervals and may detect the number of doctors being connected.

FIG. 3 shows a flowchart illustrating that the number of doctors being logged in is updated when the doctor terminal 200 logs in and out. In consideration of the case where the connection with a doctor terminal 200x is unexpectedly lost, the number-of-doctors detection module 321 may check whether or not the communication with the doctor terminals that should be connected at regular intervals and may update the number of doctors being connected.

As described above, the present invention relates to a screen sharing system, a screen sharing method, and a screen sharing program for remote medical care to appropriately change the doctor's hourly wage depending on the number of doctors who are communicative to the screen sharing system through which a doctor provides remote medical care to a patient by sharing a screen with the patient.

### Doctor's Hourly-Wage Changing Process Based on Detection of Number of Doctor and Time Period

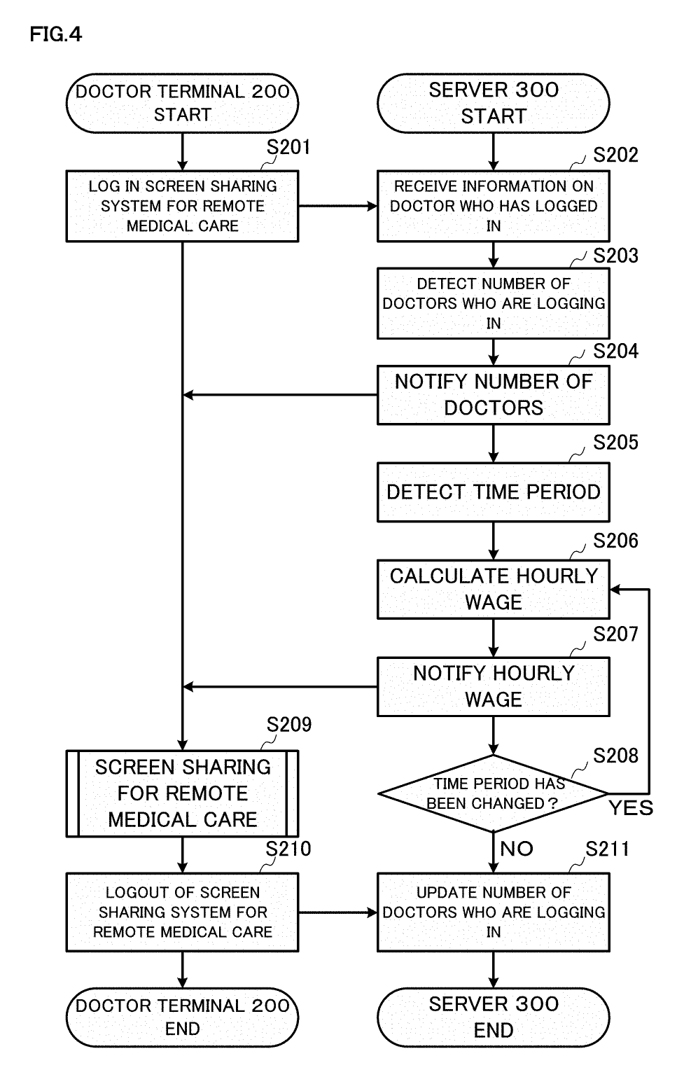

FIG. 4 shows a flow chart of the doctor's hourly-wage changing process based on the detection of the number of doctors and the time period, which is performed by the doctor terminal 200 and the server 300. The tasks executed by the modules of each of the above-mentioned units will be explained below together with this process. The patient terminal 100, the doctor terminal 200, and the server 300 can be connective among these through a communication network 400. The communication network 400 may be a public line network such as the Internet or a private line network. Since the process from the step S201 to S204 shown in FIG. 4 corresponds to that from the step S101 to S104 shown in FIG. 3, the process from the step S205 will be described below.

After the number-of-doctors detection module 321 detects the number of doctors, the doctor's hourly-wage changing module 322 detects the time period (Step S205). In this step, after the server 300 acquires the current time by using its own clock function or a network, the number-of-doctors detection module 321 may detect which time period the current time belongs to or may store only the current time to check which time period the current time belongs to in the following step S206.

Then, the doctor's hourly-wage changing module 322 calculates the doctor's hourly wage based on the number of doctors who are logged in the screen sharing system for remote medical care that is detected by the number-of-doctors detection module 321 and the time period (Step S206).

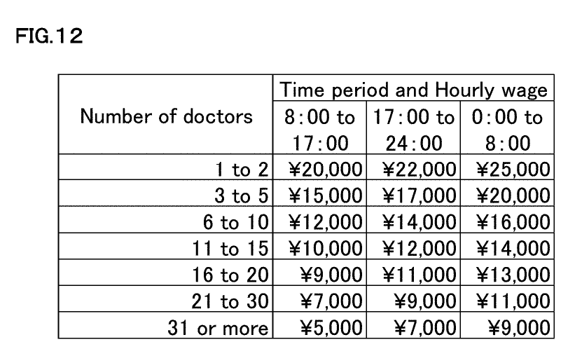

FIG. 12 shows an example illustrating the correspondence of the number of doctors to hours. The server 300 is assumed to store data that show the correspondence of the number of doctors and the time period to an hourly wage in the memory unit 310. If the number of doctors who are being connected and able to provide medical care and the current time are provided, the doctor's hourly wage can be calculated by looking up the table shown in FIG. 12. If the number-of-doctors detection module 321 detects seven doctors who can provide medical care and if the doctor's hourly-wage changing module 322 detects that the current time belongs to the time period 00:00 to 08:00, the doctor's hourly-wage changing module 322 determines the doctor's hourly wage as 16,000 yen depending on the number of doctors and the time period. If the number-of-doctors detection module 321 detects twenty doctors who can provide medical care and if the doctor's hourly-wage changing module 322 detects that the current time belongs to the time period 08:00 to 17:00, the doctor's hourly-wage changing module 322 determines the doctor's hourly wage as 9,000 yen depending on the number of doctors and the time period. Since the data shown in FIG. 12 is just an example, the system operator may appropriately set how to divide time period depending on the system. The correspondence of the number of doctors and a time period to an hourly wage applied to the system may be changed depending on the number of all the doctors registered in the screen sharing system for remote medical care. The doctor's hourly-wage changing module 322 may automatically create the correspondence table of the number of doctors and a time period to an hourly wage based on the budget allocated to the doctors' wages. Furthermore, the doctor's hourly-wage changing module 322 may mechanically learn the settings of the correspondence of the number of doctors and a time period to an hourly wage when the rate of doctors who are working on the system is high, when a patient is more likely to receive medical care immediately, and when the doctor's wage decreases, and may calculate based on the result.

Then, the doctor's hourly-wage changing module 322 notifies the hourly wage to the doctor terminal 200 (Step S207). The notified hourly wage may be used when the hourly wage is confirmed on the doctor terminal 200. The doctor's hourly-wage changing module 322 may notify the hourly wage to the doctor terminals 200a to 200n other than the doctor terminal 200 that are logged in the screen sharing system for remote medical care, at the same time. FIG. 4 shows a flowchart illustrating that the doctor's hourly-wage changing module 322 of the server 300 voluntarily notifies the hourly wage. However, the doctor's hourly-wage changing module 322 may notify the hourly wage in response to a query about the hourly wage from the doctor terminal 200. If the hourly wage changes every a few minutes, the calculation of the wage of the doctor 600 is complicated. Therefore, the update timing of the hourly wage to the doctor terminal 200 may be set in the system at every preferable period, for example, 10, 30, or 60 minutes.

The doctor's hourly-wage changing module 322 checks whether or not the time period has been changed (Step S208). In an example shown in FIG. 12, the result of the step S208 is YES at 08:00 and 17:00. If the time period has been changed, the process returns to the step S206, calculates the hourly wage (Step S206) again, and notifies the hourly wage to the doctor terminal 200 (Step S207). If the time period has not been changed, the process proceeds to the following step.

Then, the doctor terminal 200 performs the screen sharing process for remote medical care with the patient terminal 100 (Step S209). The screen sharing process for remote medical care will be described later.

After the screen sharing process for remote medical care ends, the doctor terminal 200 transmits instruction to log out of the screen sharing system for remote medical care to the server 300 through the communication unit 260 (Step S210).

Finally, the server 300 receives information on the doctor who has logged out through the communication unit 330. Then, the number-of-doctors detection module 321 updates the number of doctors who are logged in the screen sharing system for remote medical care (Step S211). At this point, the number-of-doctors detection module 321 performs a process to decrease the number of doctors being connected by the number of doctor terminals 200 that has been disconnected by logout. At this time, in consideration of the case where the connection with a logged in doctor terminal 200x is unexpectedly lost, the number-of-doctors detection module 321 may check whether or not the communication with the doctor terminals that should be connected are alive at regular intervals and may detect the number of doctors being connected.

FIG. 4 shows a flowchart illustrating that the number of doctors being logged in is updated when the doctor terminal 200 logs in and out. In consideration of the case where the connection with a doctor terminal 200x is unexpectedly lost, the number-of-doctors detection module 321 may check whether or not the communication with the doctor terminals that should be connected are alive at regular intervals and may update the number of doctors being connected.

As described above, the present invention relates to a screen sharing system, a screen sharing method, and a screen sharing program for remote medical care to appropriately change the doctor's hourly wage depending on the number of communicative doctors and the time period in a screen sharing system through which a doctor provides remote medical care to a patient by sharing a screen with the patient.

### Screen Sharing Process for Remote Medical Care

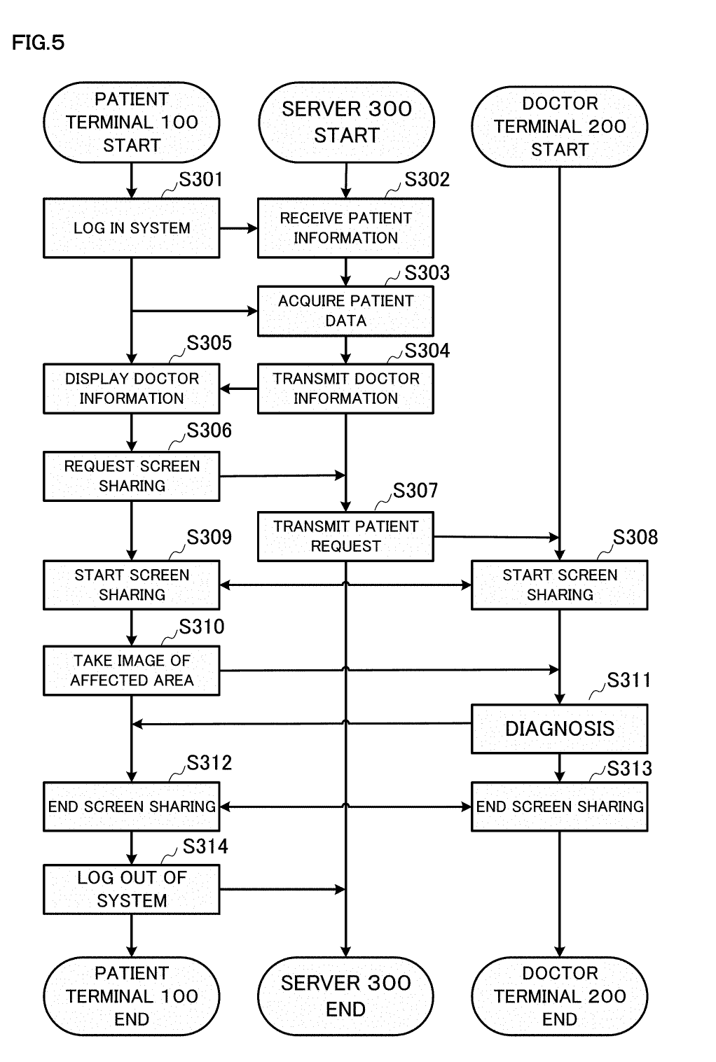

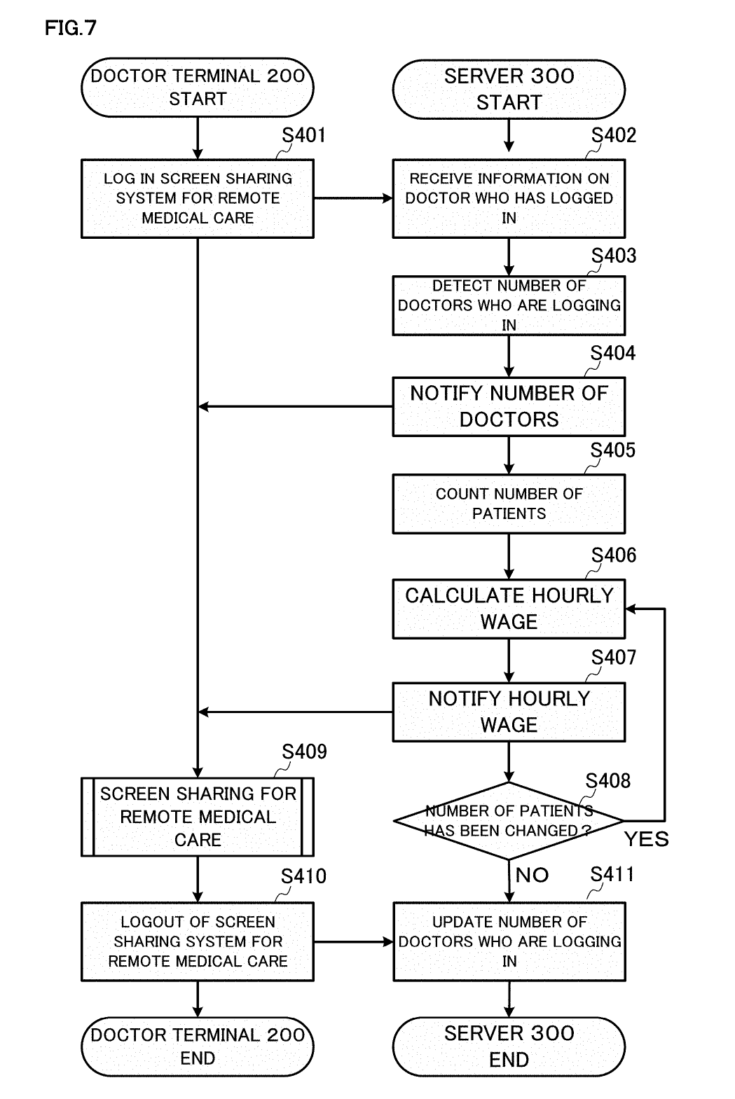

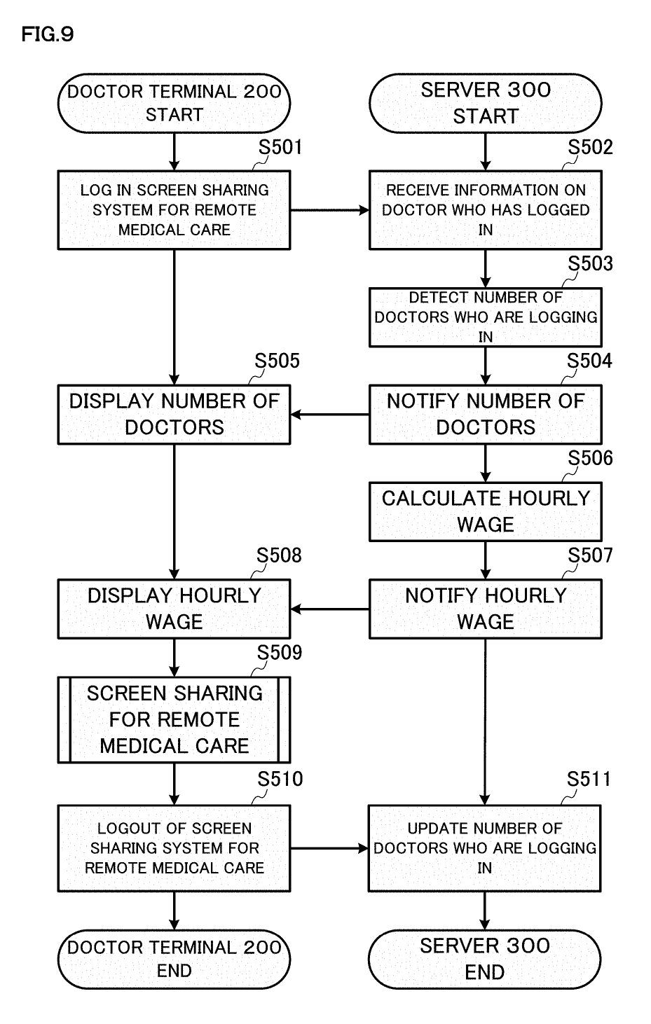

FIG. 5 shows a flow chart of the screen sharing process for remote medical care performed by the patient terminal 100, the doctor terminal 200, and the server 300. The step S107 shown in FIG. 3 and the step S209 shown in FIG. 4 correspond to the step S409 shown in FIG. 7 and the step S509 shown in FIG. 9, respectively, which will be described later. The doctor terminal 200 is assumed to log in the screen sharing system for remote medical care before the start of the flow chart shown in FIG. 5.

First, the patient terminal 100 transmits patient information necessary to log in the server 300 through the communication unit 160 and then logs in the screen sharing system for remote medical care (Step S301). The patient information herein consists of account information including a name, a nickname, and a patient ID, authentication information including a password, a passphrase, an IC card, a smart card, a fingerprint, and an iris. The flow chart shown in FIG. 3 illustrates that the login is succeeded for shorthand. However, if the login fails, the login process may suggest to the doctor to try the login again. If the login fails a certain number of times, the login process may freeze the account and then ends.

Then, the server 300 receives patient information on the patient who has newly logged in through the communication unit 330 (Step S302). The server 300 receives not only patient information but also information necessary to check the connection with the patient terminal 100 in the following process from the patient terminal 100.

Then, the control unit 320 of the server 300 acquires patient data from the patient terminal 100 or the memory unit 310 based on the received patient information (Step S303). The patient data consists of information necessary for medical care, including a name, an age, a sex, a desired clinical department, a medical care type, and the doctor in charge and the date and time when the patient used the screen sharing system for remote medical care before. The type of medical care herein includes re-examination, initial visit, and reservation for medical care. The patient data may include the description of the symptom as needed.

The control unit 320 of the server 300 generates doctor information on each of the doctors who are connecting to the system and can provide medical care based on the patient information and the patient data and transmits the doctor information to the patient terminal 100 (Step S304). The doctor information herein includes a doctor's name, profile, photograph, and clinical department which are necessary to select a doctor by the patient terminal 100. Furthermore, the number of times that the patient 500 of the patient terminal 100 had an examination from each doctor in the past may be added. The doctor information on each doctor may be stored in a database provided in the memory unit 310 of the server 300.

The patient terminal 100 displays doctor information on the output unit 130 based on the received doctor information (Step S305).

In response to the selection of a doctor by the patient 500 through the input unit 120, the control unit 150 of the patient terminal 100 transmits a request to share a screen with the doctor terminal 200 of the selected doctor 600 to the server 300 (Step S306).

The server 300 receives this screen sharing request and transmits a patient request to the desired doctor terminal 200 (Step S307). At this point, in addition to the screen sharing request from a patient, the server 300 may transmits information including the patient's name, sex, age, desired clinical department, medical care type, past usage, and the description for the symptom to the doctor terminal 200.

The control unit 250 of the doctor terminal 200 receives the screen sharing request from the patient terminal 100 and starts screen sharing (Step S308). For shorthand, FIG. 5 shows a flowchart merely illustrating that the screen sharing request can be received and then screen sharing can be started, immediately. However, if the doctor terminal 200 does not respond to the patient request within a certain time or if rejecting the patient request, the server 300 may return to the doctor information transmitting process of the step S304, notify the patient terminal 100 that the selected doctor cannot respond to the screen sharing request, and transmit updated doctor information to the patient terminal 100 to have the patient to select a doctor again. Furthermore, if the selected doctor terminal 200 is sharing a screen with another patient terminal 100x, the output unit 130 of the patient terminal 100 may output the option of waiting for the end of the screen sharing or making a reservation at a specified time. In this case, the estimated waiting time may also be displayed.

When the doctor terminal 200 receives the screen sharing request, the control unit 150 of the patient terminal 100 starts to share a screen (Step S309). The images taken by the camera units 110 and 210 are output on the patient terminal 100 and the doctor terminal 200 together with voices during the screen sharing. The screen sharing method is not limited to the scope of the present invention. Any existing technologies are applicable.

After the start of the screen sharing, an image of the affected area is taken by the camera unit 110 of the patient terminal 100 and screen-shared with the doctor terminal 200 (Step S310). Taking the image may be instructed from the doctor terminal 200.

The doctor 600 checks the image of the affected area through the output unit 230 of the doctor terminal 200, diagnoses the patient 500, and tells the result through the patient terminal 100 (Step S311). In the diagnosis output to the output unit 130 of the patient terminal 100, an image taken by the camera unit 210 of the doctor terminal 200, the voice of the doctor 600 that is input from the input unit 220, characters input by using the input unit 220, and image data including the image of the affected area with a remark handwritten from the input unit 220 are output as needed. The steps S310 and S311 may be repeated while the patient 500 and the doctor 600 need the repeat.

After medical care, the control unit 150 of the patient terminal 100 ends the screen sharing in response to the input of the patient 500 and notifies the end to the doctor terminal 200 (Step S312).

The doctor terminal 200 also ends the screen sharing in response to the notification of the end of the screen sharing from the patient terminal 100 (Step S313). The order of the steps S312 and S313 may be reversed to have the doctor terminal 200 notify the end of screen sharing to the patient terminal 100.

Finally, the patient terminal 100 transmits instruction to log out of the screen sharing system for remote medical care to the server 300 through the communication unit 160 (Step S314).

This is the screen sharing process for remote medical care for the patient terminal 100. Even after the process ends, the doctor terminal 200 is assumed to log in the screen sharing system for remote medical care and to be able to receive a screen sharing request from another patient terminal 100. FIG. 5 shows a flowchart illustrating that the patient terminal 100 and the doctor terminal 200 are directly communicated with each other after the screen sharing process starts. However, the communication may be performed through the server 300 as needed. In this case, the server 300 may store the record of screen sharing.

Screen Sharing System for Remote Medical Care Provided with Number-of-Patients Counting Function

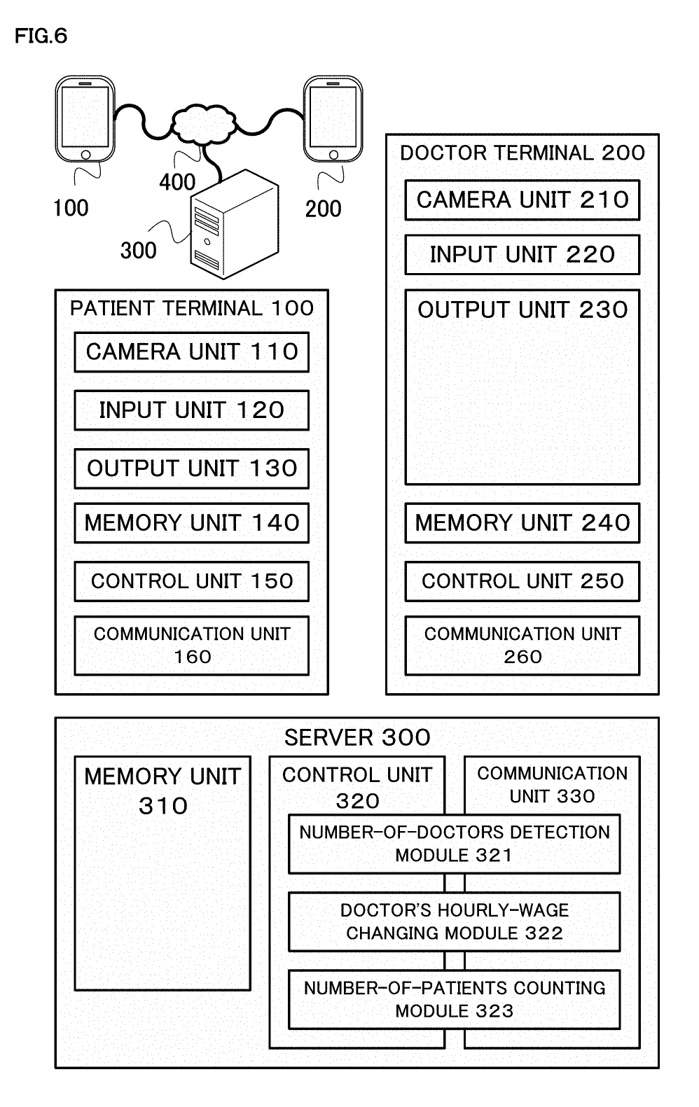

FIG. 6 shows a functional block diagram of the patient terminal 100, the doctor terminal 200, and the server 300 to illustrate the relationship among the functions if the system is provided with a number-of-patients counting function. In addition to the configuration shown in FIG. 2, the control unit 320 of the server 300 achieves a number-of-patients counting module 323 in cooperation with the communication unit 330.

FIG. 7 shows a flow chart of the doctor's hourly-wage changing process based on the detection of the number of doctors and the count of the number of the patients that is performed by the doctor terminal 200 and the server 300. The tasks executed by the modules of each of the above-mentioned units will be explained below together with this process. The patient terminal 100, the doctor terminal 200, and the server 300 can be connective among these through a communication network 400. The communication network 400 may be a public line network such as the Internet or a private line network. Since the process from the step S401 to S404 shown in FIG. 7 corresponds to that from the step S101 to S104 shown in FIG. 3, the process from the step S405 will be described below.

After the number-of-doctors detection module 321 detects the number of doctors, the number-of-patients counting module 323 counts the number of patients who are connecting with the screen sharing system for remote medical care (Step S405). At this point, the number-of-patients counting module 323 may count the number of all the patients that are connecting with the screen sharing system for remote medical care. Alternatively, the number-of-patients counting module 323 may count only the patient terminals 100 that are waiting for medical care without sharing a screen with the doctor terminal 200 as the number of patients. Which counting method is used may be appropriately set by the system operator as needed.

Then, the doctor's hourly-wage changing module 322 calculates the doctor's hourly wage based on the number of doctors who are logged in the screen sharing system for remote medical care that is detected by the number-of-doctors detection module 321 and the number of patients that is counted by the number-of-patients counting module 323 (Step S406).

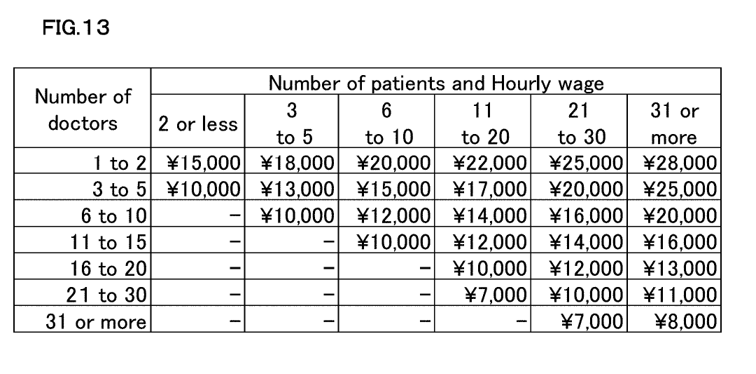

FIG. 13 shows an example illustrating the correspondence of the number of doctors and the number of patients to an hourly wage. The server 300 is assumed to store data that show the correspondence of the number of doctors and the number of patients to an hourly wage in the memory unit 310. Based on the number of doctors who are being connected and able to provide medical care and the counted number of patients, the doctor's hourly wage can be calculated by looking up the table shown in FIG. 13. If the number-of-doctors detection module 321 detects four doctors who can provide medical care and if the number-of-patients counting module 323 counts ten patients, the doctor's hourly-wage changing module 322 determines the doctor's hourly wage as 15,000 yen depending on the numbers of doctors and patients. If the number-of-doctors detection module 321 detects eight doctors who can provide medical care and if the number-of-patients counting module 323 counts five patients, the doctor's hourly-wage changing module 322 determines the doctor's hourly wage as 10,000 yen. Since the data shown in FIG. 13 is just an example, the system operator may appropriately set how to divide patients depending on the system. Furthermore, the symbol “-” shown as the number of doctors in FIG. 13 means that the doctors are too many for the patients. In this case, the screen sharing system for remote medical care may keep an additional doctor terminal 200y from logging in. The correspondence of the number of doctors and the number of patients to an hourly wage applied to the system may be changed depending on the number of all the doctors registered in the screen sharing system for remote medical care. The doctor's hourly-wage changing module 322 may automatically create the correspondence table of the number of doctors and the number of patients to an hourly wage based on the budget allocated to the doctors' wages. Furthermore, the doctor's hourly-wage changing module 322 may mechanically learn the settings of the correspondence of the number of doctors and the number of patients to an hourly wage when the rate of doctors who are working on the system is high, when a patient is more likely to receive medical care immediately, and when the doctor's wage decreases, and may calculate based on the result.

Then, the doctor's hourly-wage changing module 322 notifies the hourly wage to the doctor terminal 200 (Step S407). The notified hourly wage may be used when the hourly wage is confirmed on the doctor terminal 200. The doctor's hourly-wage changing module 322 may notify the hourly wage to the doctor terminals 200a to 200n other than the doctor terminal 200 that are logged in the screen sharing system for remote medical care, at the same time. FIG. 7 shows a flowchart illustrating that the doctor's hourly-wage changing module 322 of the server 300 voluntarily notifies the hourly wage. However, the doctor's hourly-wage changing module 322 may notify the hourly wage in response to a query about the hourly wage from the doctor terminal 200. If the hourly wage changes every a few minutes, the calculation of the wage of the doctor 600 is complicated. Therefore, the update timing of the hourly wage to the doctor terminal 200 may be set in the system at every preferable period, for example, 10, 30, or 60 minutes.

The number-of-patients counting module 323 checks whether or not the number of patients has been changed (Step S408). If the number of patients has been changed from the previous number at the timing when a patient logs in and logs out, the process returns to the step S406 to calculate the hourly wage again and notifies the hourly wage to the doctor terminal 200 (Step S407). If the number of patients has been changed, the process proceeds to the following step.

Then, the doctor terminal 200 performs the screen sharing process for remote medical care with the patient terminal 100 (Step S409). The screen sharing process for remote medical care is described above.

After the screen sharing process for remote medical care ends, the doctor terminal 200 transmits instruction to log out of the screen sharing system for remote medical care to the server 300 through the communication unit 260 (Step S410).

Finally, the server 300 receives information on the doctor who has logged out through the communication unit 330. Then, the number-of-doctors detection module 321 updates the number of doctors who are logged in the screen sharing system for remote medical care (Step S411). At this point, the number-of-doctors detection module 321 performs a process to decrease the number of doctors being connected by the number of doctor terminals 200 that has been disconnected by logout. At this time, in consideration of the case where the connection with a logged in doctor terminal 200x is unexpectedly lost, the number-of-doctors detection module 321 may check whether or not the communication with the doctor terminals that should be connected are alive at regular intervals and may detect the number of doctors being connected.

FIG. 7 shows a flowchart illustrating that the number of doctors being logged in is updated when the doctor terminal 200 logs in and out. In consideration of the case where the connection with a doctor terminal 200x is unexpectedly lost, the number-of-doctors detection module 321 may check whether or not the communication with the doctor terminals that should be connected are alive at regular intervals and may update the number of doctors being connected. Furthermore, the number-of-patients counting module 323 may check whether or not the communication with a patient terminal that should be connected are alive at regular intervals and may count the number of patients being connected

As described above, the present invention relates to a screen sharing system, a screen sharing method, and a screen sharing program for remote medical care to appropriately change the doctor's hourly wage depending on the number of patients and the number of doctors who are communicative to the screen sharing system through which a doctor provides remote medical care to a patient by sharing a screen with the patient.

Doctor's Hourly-Wage Changing Process Provide with Doctor's Hourly-Wage Display Function and Number-of-Doctors Display Function

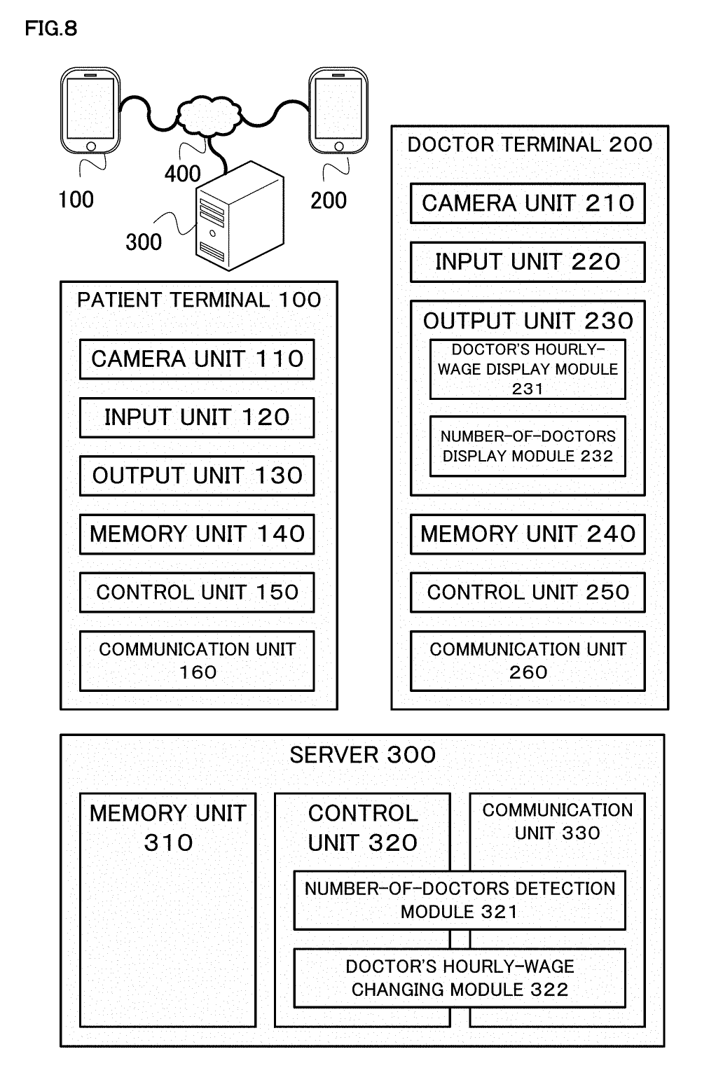

FIG. 8 shows a functional block diagram of the patient terminal 100, the doctor terminal 200, and the server 300 to illustrate the relationship among the functions if the system has a doctor's hourly-wage display function and a number-of-doctors display function. In addition to the configuration shown in FIG. 2, the output unit 230 of the doctor terminal 200 is provided with a doctor's hourly-wage display module 231 and a number-of-doctors display module 232. FIG. 8 shows an example where the output unit 230 provided with both of the doctor's hourly-wage display module 231 and the number-of-doctors display module 232. However, the output unit 230 may have only either of them.

FIG. 9 shows a flow chart of the doctor's hourly-wage changing process based on the detection of the number of doctors that is performed by the doctor terminal 200 and the server 300 if a doctor's hourly wage display process and a number-of-doctors display process are performed. The tasks executed by the modules of each of the above-mentioned units will be explained below together with this process. The patient terminal 100, the doctor terminal 200, and the server 300 can be connective among these through a communication network 400. The communication network 400 may be a public line network such as the Internet or a private line network. Since the process from the step S501 to S504 shown in FIG. 9 corresponds to that from the step S101 to S104 shown in FIG. 3, the process from the step S505 will be described below.

After the number-of-doctors detection module 321 notifies the number of doctors to the doctor terminal 200, the number-of-doctors display module 232 of the doctor terminal 200 displays the number of doctors on the output unit 230 (Step S505). The number of doctors is output by display herein but may be by voices.

Then, the doctor's hourly-wage changing module 322 calculates the doctor's hourly wage based on the number of doctors who are logged in the screen sharing system for remote medical care that is detected by the number-of-doctors detection module 321 (Step S506).

FIG. 11 shows an example illustrating the correspondence of the number of doctors to an hourly wage. The server 300 is assumed to store data that show the correspondence of the number of doctors to an hourly wage in the memory unit 310. If the number of doctors who are being connected and able to provide medical care is provided, the doctor's hourly wage can be calculated by looking up the table shown in FIG. 11. If the number-of-doctors detection module 321 detects twelve doctors who can provide medical care, the doctor's hourly-wage changing module 322 determines the doctor's hourly wage as 10,000 yen depending on the number of doctors: 12. Since the table shown in FIG. 11 is just an example, the correspondence of the number of doctors to an hourly wage that are applied to the system may be changed depending on the number of all the doctors registered in the screen sharing system for remote medical care. The doctor's hourly-wage changing module 322 may automatically create the correspondence table of the number of doctors to an hourly wage based on the budget allocated to the doctors' wages. Furthermore, the doctor's hourly-wage changing module 322 may mechanically learn the settings of the correspondence of the number of doctors to an hourly wage when the rate of doctors who are working on the system is high, when a patient is more likely to receive medical care immediately, and when the doctor's wage decreases, and may calculate based on the result.

Then, the doctor's hourly-wage changing module 322 notifies the hourly wage to the doctor terminal 200 (Step S507). The notified hourly wage may be used when the hourly wage is confirmed on the doctor terminal 200. Furthermore, the doctor's hourly-wage changing module 322 may notify the hourly wage to the doctor terminals 200a to 200n other than the doctor terminal 200 that are logged in the screen sharing system for remote medical care. FIG. 7 shows a flowchart illustrating that the doctor's hourly-wage changing module 322 of the server 300 voluntarily notifies the hourly wage. However, the doctor's hourly-wage changing module 322 may notify the hourly wage in response to a query about the hourly wage from the doctor terminal 200. If the hourly wage changes every a few minutes, the calculation of the wage of the doctor 600 is complicated. Therefore, the update timing of the hourly wage to the doctor terminal 200 may be set in the system at every preferable period, for example, 10, 30, or 60 minutes.

Then, the doctor's hourly-wage display module 231 displays the doctor's hourly wage on the output unit 230 of the doctor terminal 200 (Step S508). The number of doctors is output by display herein but may be by voices.

Then, the doctor terminal 200 performs the screen sharing process for remote medical care with the patient terminal 100 (Step S509). The screen sharing process for remote medical care is described above.

After the screen sharing process for remote medical care ends, the doctor terminal 200 transmits instruction to log out of the screen sharing system for remote medical care to the server 300 through the communication unit 260 (Step S510).

Finally, the server 300 receives information on the doctor who has logged out through the communication unit 330. Then, the number-of-doctors detection module 321 updates the number of doctors who are logged in the screen sharing system for remote medical care (Step S511). At this point, the number-of-doctors detection module 321 performs a process to decrease the number of doctors being connected by the number of doctor terminals 200 that has been disconnected by logout. At this time, in consideration of the case where the connection with a logged in doctor terminal 200x is unexpectedly lost, the number-of-doctors detection module 321 may check whether or not the communication with the doctor terminals that should be connected are alive at regular intervals and may detect the number of doctors being connected.

FIG. 9 shows a flowchart illustrating that the number of doctors being logged in is updated when the doctor terminal 200 logs in and out. In consideration of the case where the connection with a doctor terminal 200x is unexpectedly lost, the number-of-doctors detection module 321 may check whether or not the communication with the doctor terminals that should be connected are alive at regular intervals and may update the number of doctors being connected.

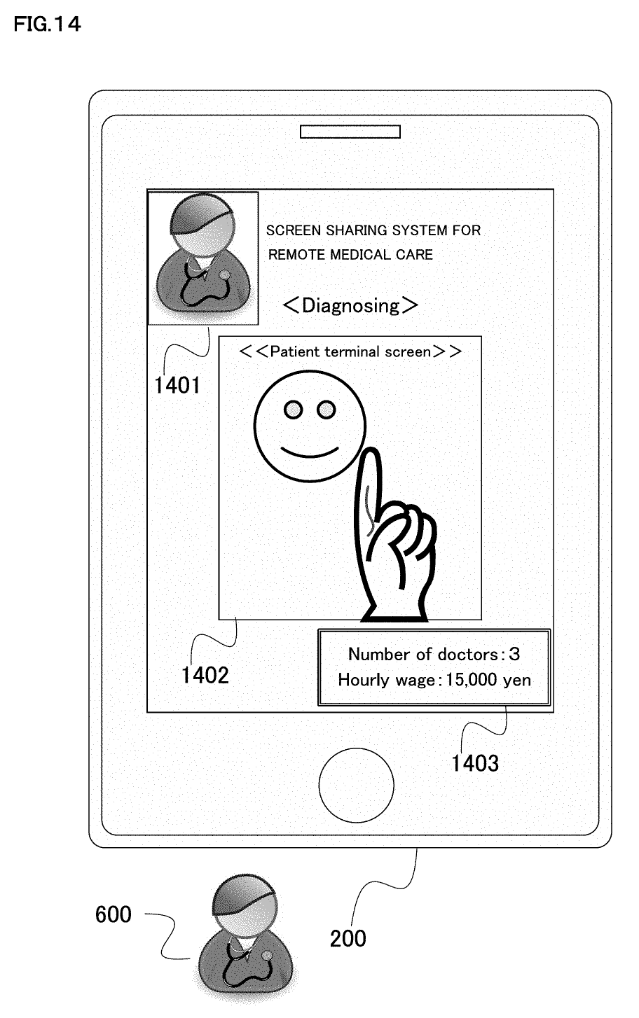

FIG. 14 shows an output example to display the number of doctors and the hourly wage on the doctor terminal 200 during medical care. The output unit 230 of the doctor terminal 200 that is sharing a screen with a patient terminal 100 outputs an image that the doctor taken with the camera unit 210 as a display 1401. Furthermore, the image of the affected area taken by the camera unit 110 of the patient terminal 100 is shared and output as a display 1402. The number-of-doctors display module 232 and the doctor's hourly-wage display module 231 display the number of doctors and the doctor's hourly wage, respectively, on the shared screen during remote medical care. The display 1403 is just one example.

FIG. 9 shows a flowchart illustrating that the number of doctors and the hourly wage are displayed after login. However, the system may have a configuration to have a doctor 600 to log in the system after the doctor 600 confirms the number of doctors and the hourly wage that have been displayed on the doctor terminal 200 since the doctor 600 requested login. In this case, the process of the steps S503 to S508 may be performed when the doctor terminal 200 requests login, and then the login process may be completed in the screen sharing system for remote medical care after the doctor 600 confirms the number of doctors and the hourly wage that are being displayed.

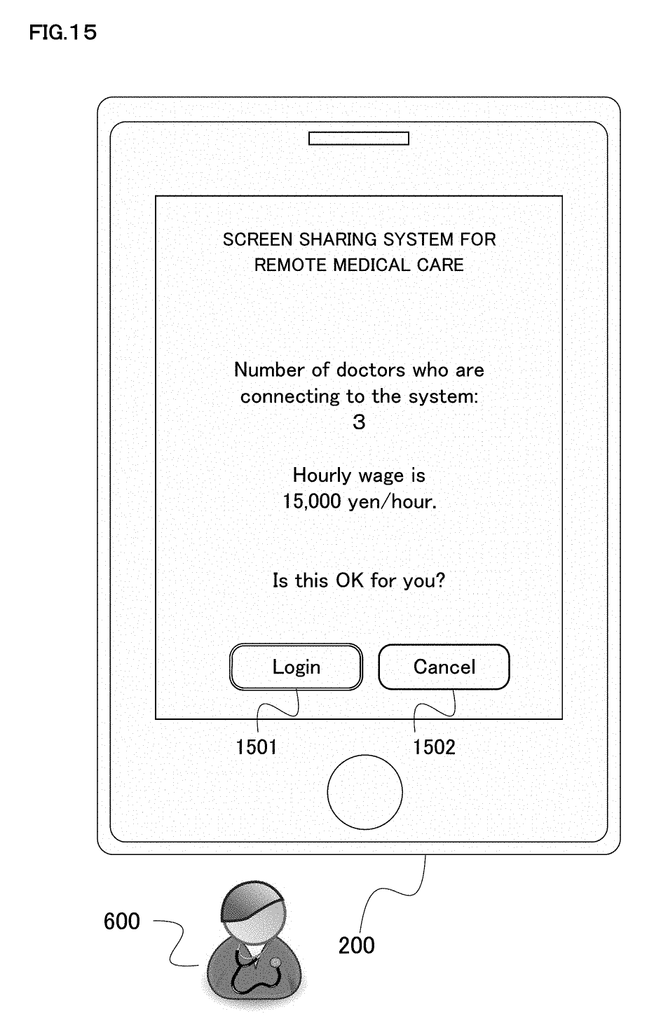

FIG. 15 shows an output example to have a doctor 600 to log in the system after the doctor 600 confirms the number of doctors and the hourly wage that are being displayed on the doctor terminal 200. The number-of-doctors display module 232 and the doctor's hourly-wage display module 231 display the number of doctors and the doctor's hourly wage, respectively, on the output unit 230 of the doctor terminal 200. If a doctor 600 selects the login button 1501, the doctor terminal 200 and the server 300 complete the login process. If a doctor 600 selects the cancel button 1502, the doctor terminal 200 and the server 300 cancels the login to the screen sharing system for remote medical care.

As described above, the present invention relates to a screen sharing system, a screen sharing method, and a screen sharing program for remote medical care to appropriately change the doctor's hourly wage depending on the number of doctors who are communicative to the screen sharing system through which a doctor provides remote medical care to a patient by sharing a screen with the patient and to display the number of doctors who are connective to the system and the doctor's hourly wage.

As described above, the present invention is to provide a screen sharing system, a screen sharing method, and a screen sharing program for remote medical care to appropriately change the doctor's hourly wage depending on the number of doctors who are connective to the system and other elements. In addition to the above-mentioned elements, the elements to make the hourly wage change may include the disease name, the symptom, and the urgency of a patient 500, and the number of times that the patient 500 used the system. Furthermore, special cases where the doctor's hourly-wage changing module 322 raises the doctor's hourly wage more than usually may be set, for example, if the patient 500 is a complainer, if the doctor 600 appreciated by patients 500, if the doctor 600 specializes in many diseases, and if the doctor 600 can speak to patients 500 in a language other than the native language.

To achieve the means and the functions that are described above, a computer (including a CPU, an information processor, and various terminals) reads and executes a predetermined program. For example, the program is provided in the form recorded in a computer-readable medium such as a flexible disk, CD (e.g., CD-ROM), DVD (e.g., DVD-ROM, DVD-RAM), or a compact memory. In this case, a computer reads a program from the record medium, forwards and stores the program to and in an internal or an external storage, and executes it. The program may be previously recorded in, for example, a storage (record medium) such as a magnetic disk, an optical disk, or a magnetic optical disk and provided from the storage to a computer through a communication line.

The embodiments of the present invention are described above. However, the present invention is not limited to the above-mentioned embodiments. The effect described in the embodiments of the present invention is only the most preferable effect produced from the present invention. The effects of the present invention are not limited to those described in the embodiments of the present invention.

### REFERENCE SIGNS LIST

100 Patient terminal
200 Doctor terminal
300 Server
400 Communication network
500 Patient
600 Doctor

1. A screen sharing system for remote medical care that changes a doctor's hourly wage based on the number of doctors who are connective to the system.

2. The screen sharing system according to claim 1 that changes a doctor's hourly wage based on a time period.

3. The screen sharing system according to claim 1 that changes a doctor's hourly wage based on the number of patients who are connecting to the system.

4. The screen sharing system according to claim 1 that displays the doctor's hourly wage on a doctor terminal.

5. The screen sharing system according to claim 1 that displays the number of doctors who are connective to the system on a doctor terminal.

6. A screen sharing method for remote medical care, comprising a step of changing a doctor's hourly wage based on the number of doctors who are connective to the system.

7. A computer program product for use in a screen sharing system for remote medical care, comprising a non-transitory computer usable medium having a set of instructions physically embodied therein, the set of instructions including computer readable program code, which when executed by the system causes the information processing unit to change a doctor's hourly wage based on the number of doctors who are connective to the system.

8. A screen sharing system for remote medical care, comprising: a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area;
a number-of-doctors detection unit that detects the number of doctors who can provide remote medical care based on the number of doctor terminals that are connective to the system; and
a doctor's hourly-wage changing unit that changes the hourly wage of the doctor based on the detected number of the doctors.

9. The screen sharing system according to claim 8, wherein the doctor's hourly-wage changing unit changes a doctor's hourly wage based on a time period.

10. The screen sharing system according to claim 8, further comprising a number-of-patients counting unit that counts the number of patients who are connecting to the system, in which the doctor's hourly-wage changing unit changes the hourly wage of the doctor based on the number of the patients.

11. The screen sharing system according to claim 8, further comprising a doctor's hourly-wage display unit that displays the doctor's hourly wage on the doctor terminal.

12. The screen sharing system according to claim 8, further comprising a number-of-doctors display unit that displays the number of the doctors on the doctor terminal.

13. A screen sharing method for remote medical care, executed by a screen sharing system for remote medical that includes a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area; comprising:
detecting the number of doctors who can provide remote medical care based on the number of doctor terminals that are connective to the system; and
changing the hourly wage of the doctor based on the detected number of the doctors.

14. A computer program product for use in a screen sharing system for remote medical care that includes a patient terminal and a doctor terminal, the patient terminal being connected with the doctor terminal through a network, through which a doctor provides remote medical care to a patient while sharing the screen of the patient terminal with the doctor terminal, the patient terminal taking and displaying the image of an affected area, comprising a non-transitory computer usable medium having a set of instructions physically embodied therein, the set of instructions including computer readable program code, which when executed by the system causes the information processing unit to
detect the number of doctors who can provide remote medical care based on the number of doctor terminals that are connective to the system; and
change the hourly wage of the doctor based on the detected number of the doctors.
